(19) United States
(12) Patent Application Publication
(10) Pub. No.: US 2004/0138507 A1
(43) Pub. Date: Jul. 15, 2004

(54) Process for production of oxygenated organic compounds

(76) Inventors: Frank Alfred Kirk, York (GB); David Hugh Miles, Middlesbrough (GB); Gordon Howard Whitfield, Middlesbrough (GB)

Correspondence Address: RATNERPRESTIA, P O BOX 980, VALLEY FORGE, PA, 19482-0980, US

(21) Appl. No.: 10/471,961

(22) PCT Filed: Mar. 13, 2002
(86) PCT No.: PCT/GB02/01010

(30) Foreign Application Priority Data
Mar. 14, 2001 (GB) GB0106219.9

(51) Int. Cl. C07C 45/90

(52) U.S. Cl. 568/451

(57) ABSTRACT

A process for the hydroformylation of olefins to produce oxygenated compounds comprises reacting together an olefin, carbon monoxide and hydrogen at elevated temperature and pressure in the presence of a rhodium-containing hydroformylation catalyst and an amount of cobalt or a cobalt compound not exceeding that corresponding to 100 ppm w/w of cobalt metal on the olefin. The reaction is carried out homogeneously and in the absence of a phosphorus-containing organic ligand. The addition of cobalt or a cobalt compound facilitates the reaction of less reactive olefin feedstocks or feedstocks which contain certain impurities.

This invention relates to the production of oxygenated organic compounds and in particular to the production of aldehydes and alcohols by hydroformylation of olefins.

Olefins may be reacted with carbon monoxide and hydrogen at elevated pressures and elevated temperatures in the presence of a catalyst to produce aldehydes and alcohols. This reaction is known as hydroformylation. The aldehyde produced by the hydroformylation reaction may be subsequently hydrogenated to the corresponding alcohol.

The hydroformylation catalyst may be rhodium or a rhodium compound. However, rhodium is a very expensive material and therefore in order to render the hydroformylation process using a rhodium-containing catalyst economical it is very desirable that the concentration of the catalyst with reference to the olefin is maintained as low as possible. Unfortunately, low concentrations of a rhodium-containing catalyst may not catalyse the hydroformylation of olefins effectively, particularly when the feedstock contains impurities which inhibit the reaction. For instance, there may be a long induction period before the hydroformylation reaction commences and if it does commence the rate of reaction may be very slow and completely uneconomical.

Furthermore, we have found that when certain olefin feedstocks are used, the reaction may proceed very slowly if at all. This can be a particular problem with highly branched feedstocks which may be unreactive due to the high degree of branching in the molecules.

We have now found that these disadvantages of the hydroformylation process using a rhodium-containing catalyst can be overcome by carrying out the process in the presence of cobalt or a cobalt compound in addition to the rhodium-containing catalyst.

U.S. Pat. No. 4,306,086, U.S. Pat. No. 4,200,592 and U.S. Pat. No. 4,599,323 describe a liganded rhodium catalyst system for the hydroformylation of propene and of a process for stabilising a rhodium/triaryl phosphine catalyst system by the addition of a cobalt compound to the catalyst complex. These liganded rhodium catalysts are used mainly in the hydroformylation of terminally unsaturated, linear olefins. Relatively low pressures and relatively high concentrations of rhodium are typically used in these reactions. This type of catalyst system may be significantly less active for the conversion of branched olefin materials or olefins with non-terminal double bonds. EP-A-0183451, EP-A-0238331 and U.S. Pat. No. 4,262,147 all describe the use of supported catalysts comprising cluster compounds of Rh and Co compounds supported on an amine resin. The hydroformylation reactions described are one-step processes, leading directly to alcohols.

According to the present invention, we provide a process for the hydroformylation of olefins to produce oxygenated compounds which comprises reacting together an olefin, carbon monoxide and hydrogen at elevated temperature and pressure in the presence of a homogeneous rhodium-containing hydroformylation catalyst and an amount of cobalt or a cobalt compound not exceeding that corresponding to 100 ppm by weight of cobalt metal on the olefin, and in the absence of a phosphorus-containing organic ligand.

The reaction proceeds in the absence of a phosphorus-containing organic ligand. The liganded rhodium catalyst systems, which are described in the prior art, contain an organic phosphorus-containing ligand, which is usually a triaryl phosphine compound. These reaction systems generally proceed at relatively low pressures and use a relatively large concentration of rhodium, typically of the order of 300 ppmw. The high concentration of rhodium is required because the presence of the phosphorus-containing ligand moderates the catalytic activity of the rhodium, which may be required to improve the control of the reaction process. These systems have been found to be much less active when the olefin is less reactive to hydroformylation, which is the case for branched olefins, for example.

In a second aspect of the invention, we provide a process for the hydroformylation of olefins to produce oxygenated compounds which comprises reacting together an olefin, carbon monoxide and hydrogen at elevated temperature and pressure in the presence of a rhodium-containing hydroformylation catalyst, wherein said rhodium-containing catalyst is present in an amount sufficient to provide from 1 to 20 ppm by weight of rhodium relative to the amount of olefin, and an amount of cobalt or a cobalt compound sufficient to provide from 1 to 40 ppm w/w of cobalt metal relative to the amount of the olefin.

A wide range or cobalt compounds may be used in the process of the invention, for example an oxide or a salt. It is preferred to use a cobalt compound which is soluble in the olefin or the hydroformylation reaction mixture such as, for example, a cobalt carbonyl, a cobalt hydrocarbonyl or a cobalt salt of an organic carboxylic acid, for example, naphthenic acid. It is preferred that the cobalt salt is cobalt naphthenate.

It is usually sufficient to use an amount of cobalt or a cobalt compound not exceeding the equivalent of 100 ppm w/w (parts per million by weight) cobalt metal, suitably an amount which provides from 1 to 40 ppm w/w, preferably from 5 to 30 ppmw, more preferably 10 to 25 ppmw, for example about 20 ppm w/w Co metal by weight of the olefin. It is preferred that the amount of cobalt used is such that it makes no more than a minor contribution to the catalysis of the hydroformylation reaction compared with the contribution of the rhodium-containing catalyst. At concentrations of Co above 40 ppm w/w, it is known that Co may promote undesirable side reactions which may lead to heavy impurities in the product stream. When using these preferred amounts the cobalt is, for all practical purposes, inactive as a hydroformylation catalyst.

The rhodium-containing catalyst is a homogeneous catalyst. Although a portion of the feed materials, particularly the hydrogen, may be gaseous under the reaction conditions used, the reaction is understood to take place in the liquid phase in which a part of the hydrogen is dissolved. By homogeneous, we therefore mean homogeneous with the liquid phase of the reaction mixture. The rhodium is therefore supplied to the reaction mixture in a form which is soluble in the reaction mixture under the conditions at which the reaction is performed. A preferred catalyst is a rhodium carbonyl which term includes complex carbonyls or carbonyl halides. Alternatively, the rhodium may be added to the reaction mixture in the form of finely-divided metallic rhodium or a compound of rhodium such as an oxide or a salt which is converted into a rhodium carbonyl under the conditions of the hydroformylation reaction. It is believed that a rhodium carbonyl is the active catalyst. It is preferred that the rhodium compound is a salt of an organic carboxylic acid, preferably at least a C₄ carboxylic acid, e.g. a C₄-C₃₀ carboxylic acid, more preferably a C₆-C₂₂ carboxylic acid, provided that the rhodium salt is soluble in the reaction medium, such as naphthenic, oleic or stearic acid or a rhodium carbonyl complex such as (acetylacetonato) dicarbonyl rhodium (I).

It is preferred to introduce the rhodium containing catalyst into the hydroformylation reaction zone as a suspension or a solution in an organic solvent. Suitable organic solvents are the olefin to be hydroformylated, the product or a portion of the product of the hydroformylation reaction before or after hydrogenation of the aldehydes to alcohols or a substance such as a saturated hydrocarbon which is inert under the conditions of the hydroformylation reaction.

Although larger amounts of the rhodium-containing catalyst may be used, the process of the invention is of particular value when the amount of the rhodium-containing catalyst does not exceed 20 ppm w/w, e.g. from 1 to 20 ppmw of rhodium by weight of olefin and is preferably from about 2 to about 15 ppm w/w of rhodium or a suitable amount of a rhodium compound by weight of the olefin to provide a rhodium concentration within the above-mentioned ranges. The amount of rhodium may be varied according to the feedstock so that less rhodium e.g. about 2-3 ppm w/w may be sufficient to catalyse linear olefins, whilst larger amounts are necessary for less reactive feedstocks. The preferred amounts of the rhodium-containing catalyst may very suitably be used with 1-40, preferably 5-30, more preferably 10 to 25 ppm w/w cobalt by weight of the olefin. The amount of rhodium used may exceed or be less than the amount of cobalt. The atomic ratio Rh to Co preferably lies within the range 1:0.45-70, more preferably 1:0.6-22.

The process of the invention is suitable for the hydroformylation of a wide variety of olefins such as propylene, butenes, 2-methylpentene-1, heptenes, di-isobutylene, propylene trimer, propylene tetramer and butene trimer. Mixtures of olefins may be hydroformylated. The process of the invention is applicable to the hydroformylation of olefins containing dienes, sulphur compounds or other substances which tend to inhibit the catalysis of the hydroformylation reaction by a rhodium-containing catalyst. It is also useful for the hydroformylation of olefins which are intrinsically less reactive due to their molecular structure. Thus the process of the invention is particularly applicable to the hydroformylation of commercial mixtures of heptenes, propylene trimer and olefins produced by the cracking of petroleum wax or propylene tetramer and butene trimer produced by the oligomerisation of propylene and butene respectively over a catalyst such as phosphoric acid for example.

The process of the invention may be carried out in the presence of a solvent for the olefin and the olefin may be introduced into the reaction zone as a solution in the solvent. If the olefin is a vapour under the conditions of the hydroformylation process it is preferred to carry out the process in the presence of a solvent. Suitable solvents are the product, or a portion of the product, of the hydroformylation process before or after hydrogenation, or solvents such as saturated hydrocarbons which are inert under the conditions of the hydroformylation process. The hydroformylation process of the invention is operated under elevated pressure and at elevated temperature. Suitable pressures are within the range 100 to 400 atmospheres, preferably within the range 150 to 300 atmospheres. Suitable temperatures are within the range 80° to 250° C. The carbon monoxide and hydrogen necessary to effect the hydroformylation reaction may be used in a wide range of volume ratios, for example from 10:1 to 1:10. It is, however, preferred to use carbon monoxide and hydrogen in a volume ratio within the range 3:1 to 1:3, for example 1:1. On completion of the hydroformylation process the reaction product may be hydrogenated to convert the product aldehydes into the corresponding alcohols.

### EXAMPLE 1

Except as otherwise stated, in each experiment an amount of rhodium stearate containing rhodium equivalent to 4 ppm w/w by weight of the olefin was dissolved in the olefin which was then hydroformylated in a rocked autoclave at 170° C. using a mixture of equal volumes of carbon monoxide and hydrogen under 250 atmospheres pressure. In experiments 1 and 2 the olefin was a C₆-C₈ wax-cracked alpha-olefin containing 0.04 moles dienes per kilogram while in experiment 3 it was a similar olefin containing 0.02 moles dienes per kilogram. In each experiment 90 grams of the olefin were used.

### Experiment 1

In addition to the rhodium-containing catalyst an amount of cobalt naphthenate equivalent to 20 ppm w/w by weight of cobalt on the olefin was dissolved in the olefin before carrying out the hydroformylation reaction. Absorption of gas was complete in 100 minutes and 115 grams of product were obtained. Analysis showed this product to contain 10.0% by weight unreacted olefin, 70.3% aldehydes and 5.0% high boiling material.

When the experiment was repeated using either only the rhodium-containing catalyst or only the cobalt naphthenate very little absorption of gas occurred and aldehydes could not be detected in the product.

### Experiment 2

Experiment 1 was repeated using an amount of cobalt naphthenate equivalent to 15 ppm w/w cobalt by weight of the olefin in addition to the rhodium-containing catalyst. A reaction commenced 80 minutes after setting up the experiment and absorption of gas ceased after a further 120 minutes. 112 grams product were obtained. Analysis showed this product to contain 17.0% w/w unreacted olefin, 62.0% wlw aldehyde and 4.0% high boiling material.

### Experiment 3

In addition to the rhodium-containing catalyst an amount of cobalt naphthenate equivalent to 10 ppm w/w of cobalt on the olefin was dissolved in the olefin before carrying out the hydroformylation reaction. Absorption of gas was complete in 79 minutes and 115 grams of product were obtained. Analysis showed this product to contain less than 1% w/w unreacted olefin, 75.9% w/w aldehydes and 5.0% w/w high boiling material.

When the experiment was repeated in the absence of cobalt, 90 grams of product were obtained which analysis showed to contain 65.3% w/w unreacted olefin and 15.0% w/w aldehydes.

When the experiment was repeated in the absence of rhodium and using an amount of cobalt naphthenate equivalent to 20 ppm w/w cobalt by weight of the olefin very little absorption of gas took place over 100 minutes and aldehydes could not be detected in the product.

### EXAMPLE 2

In the following experiments 240 grams of a commercial mixture of heptenes were injected by means of a gas consisting of an equimolar mixture of hydrogen and carbon monoxide into a stainless steel rocking autoclave maintained at a temperature of 170° C. The pressure in the autoclave was then raised to about 200 atmospheres by adding more gas and the autoclave rocked for about 30 minutes to obtain a steady pressure and temperature. 60 grams of the heptenes containing a rhodium-containing hydroformylation catalyst and/or cobalt compound were then injected into the autoclave and the pressure rapidly adjusted to 250 atmospheres. Pressure readings were then taken at 2 minute intervals for 45 minutes and the pressure rapidly readjusted to 250 atmospheres when it fell to 230 atmospheres by injection of more gas.

The rhodium-containing hydroformylation catalyst, used in Experiment Nos. 1, 2, 3, 4, 6 and 7 was rhodium stearate in an amount corresponding to 3.5 ppm w/w of rhodium on the olefin. In experiments Nos. 4, 5 and 7 an amount of cobalt naphthenate corresponding to 20 ppm w/w of cobalt on the olefin was used.

In Experiment No.2 the commercial mixture of heptenes contained 0.4% w/w of isoprene. In Experiments Nos. 3, 4, and 5 it contained 0.6% w/w of dicyclopentadiene while in Experiments Nos. 6 and 7 it contained 0.01 % sulphur as carbon disulphide.

The total pressure drops recorded in each experiment are shown in Table 1. A pressure drop of about 380 atmospheres is equivalent to complete conversion of the olefin.

TABLE 1

| | | | Total Pressure |
|---|---|---|---|
| Experiment | Rhodium | Cobalt | Drop |
| No. | present | present | Atmospheres |
| 1 | Yes | No | 250 |
| 2 | Yes | No | Nil |
| 3 | Yes | No | 47 |
| 4 | Yes | Yes | 188 |
| 5 | No | Yes | Nil |
| 6 | Yes | No | 82 |
| 7 | Yes | Yes | 223 |

Comparison of the pressure drops obtained in Experiments Nos. 2, 3 and 6 with the pressure drop obtained in Experiment No. 1 shows the harmful effect of dienes and sulphur compounds on the hydroformylation reaction. In these experiments no cobalt was present. Experiments Nos. 4, and 7 show how this harmful effect can be overcome by carrying out the reaction according to the process of the invention, i.e. in the presence of cobalt while the comparison of Experiments Nos. 4 and 5 shows that cobalt by itself was ineffective in promoting the hydroformylation reaction.

### EXAMPLE 3

Three experiments were carried out in which commercially available di-isobutene was hydroformylated in the matter described in Example 2 using an amount of rhodium stearate corresponding to 3.4 ppm w/v of rhodium on the di-isobutene. The reaction time was 27 minutes.

In the first experiment the total pressure drop was 195 atmospheres.

In the second experiment the di-isobutene contained 0.3% w/w of dicyclopentadiene and the total pressure drop was only 33 atmospheres.

In the third experiment the di-isobutene contained 0.3% w/w of dicyclopentadiene as in the second experiment and also cobalt naphthenate equivalent to 20 ppm w/v of cobalt on the olefin. The total pressure drop was 157 atmospheres showing the effect of the cobalt.

### EXAMPLE 4

Rhodium stearate was dissolved in propylene trimer to give a solution containing the equivalent of 4 ppm w/v of rhodium metal and the solution continuously fed at a rate of 0.51 volumes per volume of reactor space per hour, together with a mixture of equal volumes of carbon monoxide and hydrogen into a reactor maintained at a temperature of 160° C. and a pressure of 250 atmospheres. The liquid product from the reactor contained 59.6% w/w of oxygenated compounds and 40.2% w/w unreacted olefin.

In a similar experiment carried out under the same conditions except that the propylene trimer in addition to the rhodium stearate contained cobalt naphthenate equivalent to 10 ppm w/v cobalt metal, the liquid product from the reactor contained 70.0% w/w of oxygenated compounds and 29.0% w/w unreacted olefin.

### EXAMPLE 5

The following experiments demonstrate the effect of the presence of a cobalt compound on the hydroformylation of olefins using a rhodium-containing catalyst when the olefins contain a sulphur compound.

A sulphur compound was added to a commercially available mixture of heptenes and 280 grams of the heptenes was then hydroformylated in a 1-litre stainless steel stirred autoclave at 165° C. and 250 atmospheres pressure using a mixture of equal volumes of hydrogen and carbon monoxide. In each experiment the reaction time was 90 minutes. Rhodium stearate was used as the catalyst and the cobalt compound was cobalt naphthenate.

Table 2 gives the remaining reaction conditions and the conversion of heptenes to oxygenated products achieved.

TABLE 2

| | | | | | % |
|---|---|---|---|---|---|
| | | ppm w/w | ppm w/w | ppm w/w | Conversion |
| Expt | Additive | additive | rhodium | cobalt | of heptenes |
| 1 | Ethane thiol | 10 | 4 | 10 | 53 |
| 2 | | | | Nil | 6 |
| 3 | di-n-butyl | 8 | 4 | 10 | 65 |
| 4 | disulphide | | | Nil | 46 |
| 5 | di-isobutyl | 270 | 4 | 10 | 62.5 |
| 6 | disulphide | | | Nil | 16 |
| 7 | Hydrogen | 10 | 5 | 10 | 64.4 |
| 8 | sulphide | | | Nil | 51.9 |
| 9 | Carbonyl | 10 | 5 | 10 | 64 |
| 10 | sulphide | | | Nil | 52 |

### EXAMPLE 6

A series of hydroformylation reactions was carried out on a ‘butene trimer’, prepared by the oligomerisation of mixed butenes over a phosphoric acid catalyst followed by distillation to give a predominantly C₁₂ branched olefin.

In each case, a quantity of the rhodium compound (acetylacetonato)dicarbonylrhodium(I) was dissolved in the butene trimer to give a concentration equivalent to 12 ppm w/v of rhodium metal.

### Experiment 6A

The rhodium-containing butene trimer (150 ml) was hydroformylated in a 300 ml stirred autoclave at 170° C. in the presence of equal volumes of carbon monoxide and hydrogen at a total pressure of 230 bar for 4 hours. The autoclave was then cooled, the pressure released and the product was discharged for analysis.

### Experiments 6B and 6C

Two further hydroformylation experiments were carried out. As before the butene trimer contained 12 ppm w/v of rhodium but, in addition, cobalt naphthenate was added (dissolved in butene trimer feedstock) to give a cobalt concentration of 5 ppm w/v (Experiment 6B) and 20 ppm w/v (Experiment 6C). The hydroformylations were carried out under the same conditions of temperature, pressure and time as Experiment 6A.

The conversion in the three experiments was expressed as the concentration by weight of aldehyde and other oxygenated compounds in the products. The results are shown Table 3:

TABLE 3

| | Rhodium | | |
|---|---|---|---|
| Experiment | (ppm w/v) | Cobalt (ppm w/v) | Conversion (% w/w) |
| 6A | 12 | — | 1 |
| 6B | 12 | 5 | 29 |
| 6C | 12 | 20 | 58 |

1. A process for the hydroformylation of olefins to produce oxygenated compounds which comprises reacting together an olefin, carbon monoxide and hydrogen at elevated temperature and pressure in the presence of a homogeneous rhodium-containing hydroformylation catalyst and an amount of cobalt or a cobalt compound not exceeding that corresponding to 100 ppm by weight of cobalt metal on the olefin, and in the absence of a phosphorus-containing organic ligand.

2. A process as claimed in claim 1, wherein the cobalt compound comprises a cobalt carbonyl, a cobalt hydrocarbonyl or a cobalt salt of an organic carboxylic acid.

3. A process as claimed in claim 1 or claim 2, wherein the amount of cobalt or cobalt compound is an amount which provides between 1-40 ppm w/w of cobalt by weight of the olefin.

4. A process as claimed in any of the preceding claims wherein the amount of the rhodium-containing catalyst is such as to provide from 1 to 20 ppm w/w of rhodium by weight of the olefin.

5. A process as claimed in any of the preceding claims wherein the olefin comprises propylene, butenes, 2-methylpentene-1, heptenes, di-isobutylene, propylene trimer, propylene tetramer and butene trimer.

6. A process as claimed in any of the preceding claims, wherein the olefin comprises a commercial mixture of heptenes, propylene trimer and olefins produced by the cracking of petroleum wax or a commercial mixture of propylene tetramer and butene trimer produced by the oligomerisation of propylene and butene.

7. A process as claimed in any of the preceding claims, wherein the olefin contains dienes, sulphur compounds or other substances which tend to inhibit the catalysis of the hydroformylation reaction by a rhodium-containing catalyst.

8. A process for the hydroformylation of olefins to produce oxygenated compounds which comprises reacting together an olefin, carbon monoxide and hydrogen at elevated temperature and pressure in the presence of a rhodium-containing hydroformylation catalyst, wherein said rhodium-containing catalyst is present in an amount sufficient to provide from 1 to 20 ppm by weight of rhodium relative to the amount of olefin, and an amount of cobalt or a cobalt compound sufficient to provide from 1 to 40 ppm w/w of cobalt metal relative to the amount of the olefin.

9. A process as claimed in claim 8, wherein the cobalt compound comprises a cobalt carbonyl, a cobalt hydrocarbonyl or a cobalt salt of an organic carboxylic acid.

10. A process as claimed in claim 8 or claim 9, wherein the rhodium-containing catalyst is added to the reaction mixture in the form of a rhodium salt of an organic carboxylic acid or a rhodium carbonyl complex.
